(19) United States
(12) Patent Application Publication
(10) Pub. No.: US 2025/0049334 A1
(43) Pub. Date: Feb. 13, 2025

(54) ECG ELECTRODES FOR WEARABLE PHYSIOLOGICAL MONITORS

(71) Applicant: Whoop, Inc., Boston, MA (US)

(72) Inventors: Anahis Araxy Kechejian, Arlington, MA (US); Benjamin August Rothenberg Peterson, Cambridge, MA (US); Sienna Amai Ramos, Cambridge, MA (US); Russell Austin Merrick, Wakefield, MA (US)

(21) Appl. No.: 18/798,653

(22) Filed: Aug. 8, 2024

Related U.S. Application Data
(60) Provisional application No. 63/518,273, filed on Aug. 8, 2023.

(51) Int. Cl. A61B 5/024; A61B 5/00; A61B 5/256; A61B 5/282; A61B 5/308

(52) U.S. Cl. CPC A61B 5/02416; A61B 5/256; A61B 5/282; A61B 5/308; A61B 5/6824; A61B 5/6831; A61B 2560/029; A61B 2560/0443; A61B 2560/0468; A61B 2562/0219; A61B 2562/225

(57) ABSTRACT

A wearable monitor for electrocardiography includes a first electrode for forming an electrocardiography circuit. A second electrode may be integrated into a removable strap or the like that secures the monitor in place during use. The second electrode may be coupled to internal circuitry of the monitor through one or more removable and replaceable, moving contacts that preserve an environmental seal around internal circuitry of the monitor while supporting a conductive path between the second electrode and the internal circuitry for purposes of electrocardiography. In another aspect, one of the electrodes is configured, e.g., by code executing on the monitor, for use as both an electrocardiography electrode and a user input button.

### CROSS-REFERENCE TO RELATED APPLICATIONS

This application claims priority to U.S. Provisional Pat. App. No. 63/518,273 filed on Aug. 8, 2023, the entire contents of which are hereby incorporated by reference herein.

### TECHNICAL FIELD

This disclosure generally relates to electrocardiogram electrodes for wearable physiological monitors.

### BACKGROUND

There remains a need for improved wearable physiological monitors that incorporate electrodes for capturing electrocardiograms and/or receiving user input.

### SUMMARY

A wearable monitor for electrocardiography includes a first electrode for forming an electrocardiography circuit. A second electrode may be integrated into a removable strap or the like that secures the monitor in place during use. The second electrode may be coupled to internal circuitry of the monitor through one or more removable and replaceable, moving contacts that preserve an environmental seal around internal circuitry of the monitor while supporting a conductive path between the second electrode and the internal circuitry for purposes of electrocardiography. In another aspect, one of the electrodes is configured, e.g., by code executing on the monitor, for use as both an electrocardiography electrode and a user input button

In one aspect, a wearable monitor disclosed herein may include: a housing containing circuitry and sensors for physiological monitoring; a strap configured to secure the housing in a position about a wrist of a user for use in physiological monitoring; an arm hingeably coupled to the housing and rotatable around a hinge of the housing to tighten the strap in the position about the wrist of the user, the arm including a recess; a first electrode on an exterior surface of the arm; a spring pin positioned to removably and replaceably secure the arm with the hinge in a closed position such that the strap is tightened in the position for use, the spring pin including an external contact surface for electromechanically coupling to the recess in the arm, and an electrical contact extending into an interior of the housing, the electrical contact of the spring pin conductively coupled to the external contact surface of the spring pin; a printed circuit board within the housing, the printed circuit board electrically coupled to the electrical contact of the spring pin through one or more moving contacts; a second electrode on the housing, the second electrode coupled to the printed circuit board, and the second electrode positioned to contact a skin of the user when the housing is in the position for use by the user during physiological monitoring; and electrocardiogram circuitry on the printed circuit board, the electrocardiogram circuitry coupled between the first electrode and the second electrode, and the electrocardiogram circuitry configured to capture an electrocardiogram of the user when a user contacts the first electrode with a digit on a second arm opposing a first arm where the housing is secured to the wrist by the strap.

Implementations may include one or more of the following features. The arm may be removable from and replaceable to the hinge of the housing. The housing may be waterproof. The printed circuit board may be electrically coupled to the electrical contact of the spring pin through at least one sliding contact. The printed circuit board may be electrically coupled to the electrical contact of the spring pin through at least one leaf spring. The printed circuit board may be electrically coupled to the electrical contact of the spring pin through at least one moving contact. The wearable monitor may include photoplethysmography circuitry configured to acquire heart rate data from the user through photoplethysmography when the wearable monitor is placed for use on the user.

In one aspect, a wearable monitor disclosed herein may include: a housing with an exterior surface and an interior space; electrocardiogram circuitry in the interior space of the housing; a first electrode and a second electrode. The first electrode may be positioned for access by a user when the wearable monitor is placed for use on the user, and the first electrode may be removably and replaceably coupled to the housing. The second electrode may be positioned on the exterior surface of the housing, the second electrode may be positioned to contact a skin of the user when the wearable monitor is placed for use on the user, and the second electrode may be coupled to the first electrode through the electrocardiogram circuitry in the housing.

Implementations may include one or more of the following features. The wearable monitor may include a photoplethysmography monitor. The wearable monitor may include a strap for securing the housing to the user. The wearable monitor may include a fastener configured to releasably tighten the strap to retain the wearable monitor for use on the user. The first electrode may be positioned on a surface of the fastener. The fastener and the strap may be removable from and replaceable to the housing. The fastener may have an open position and a closed position, where: in the open position of the fastener, the wearable monitor is removable from the user and the first electrode is not electrically coupled to the electrocardiogram circuitry, and in the closed position, the strap retains the housing of the wearable monitor against the skin of the user for acquisition of a physiological signal and the first electrode is electrically coupled to the electrocardiogram circuitry. The strap may include two bands that are removably and replaceably couplable to one another.

In one aspect, a computer program product disclosed herein may include computer executable code embodied in a non-transitory computer readable medium that, when executing on one or more processors of a wearable monitor, causes the wearable monitor to perform the step of monitoring two or more electrodes of a wearable monitor, where: the two or more electrodes include a first electrode and a second electrode, the second electrode is in continuous contact with a skin of a user of the wearable monitor when the wearable monitor is placed for use on the user, and the wearable monitor is configured to acquire electrocardiographic data with the two or more electrodes. The computer program product may further include computer executable code embodied in a non-transitory computer readable medium that, when executing on one or more processors of a wearable monitor, causes the wearable monitor to perform the steps of: receiving a hardware interrupt with the wearable monitor, the hardware interrupt responsive to a predetermined impedance between the first electrode and the second electrode, and the predetermined impedance indicative of user contact with the first electrode; while the hardware interrupt indicates the predetermined impedance between the first electrode and the second electrode, periodically incrementing a counter; and, in response to the counter meeting a second predetermined threshold, generating a button press signal to a process executing on the wearable monitor. Other embodiments of this aspect may include corresponding computer systems, methods, apparatus, and computer programs recorded on one or more computer storage devices, each configured to perform one or more of the aforementioned actions.

Implementations may include one or more of the following features. The wearable monitor may include a wrist-worn heart rate monitor. The computer program product may include code that, in response to the button press signal, causes the wearable monitor to perform a responsive action. The computer program product may include code that, in response to the button press signal, causes the wearable monitor to initiate an electrocardiograph acquisition. The second predetermined threshold for the counter may be selected to distinguish intentional contact with the first electrode by the user from unintentional contact with the first electrode.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system including a wearable monitor.

FIG. 7 shows a system including a wearable monitor.

FIG. 8 shows a system including a wearable monitor.

FIG. 9 shows a wearable monitor with a strap.

FIG. 10A shows a cross section of a wearable monitor.

FIG. 10B shows a cross section of a wearable monitor with an electrocardiogram (ECG) circuit path shown by way of example.

FIG. 11 shows an exploded view of a wearable monitor.

FIG. 12 shows a process for using an exposed electrode of a wearable device as a control input.

FIG. 13 shows a process for using an exposed electrode of a wearable device as a control input.

FIG. 14 is a flow chart of a method for using a physiological monitoring electrode as a user input.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended or stated purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the disclosed embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

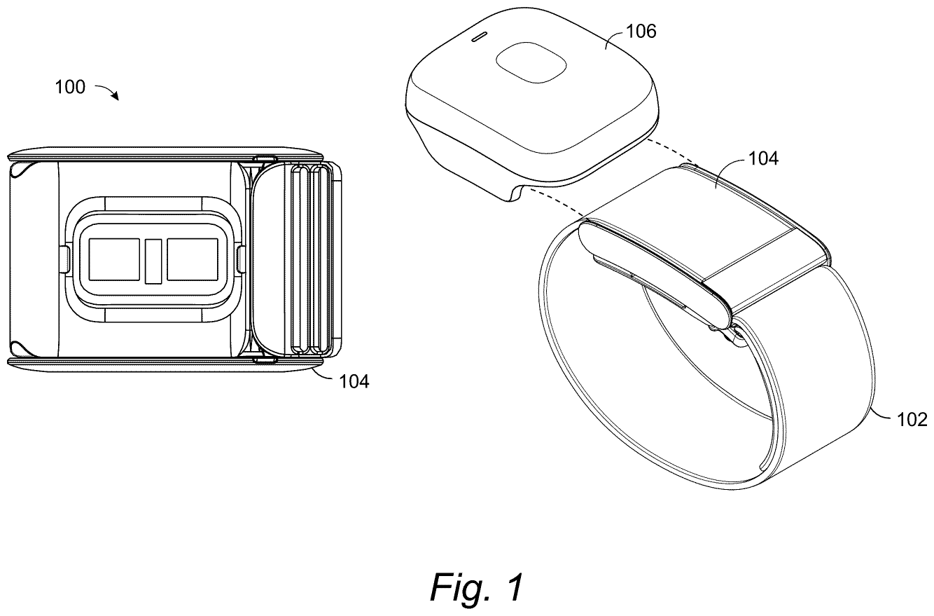

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

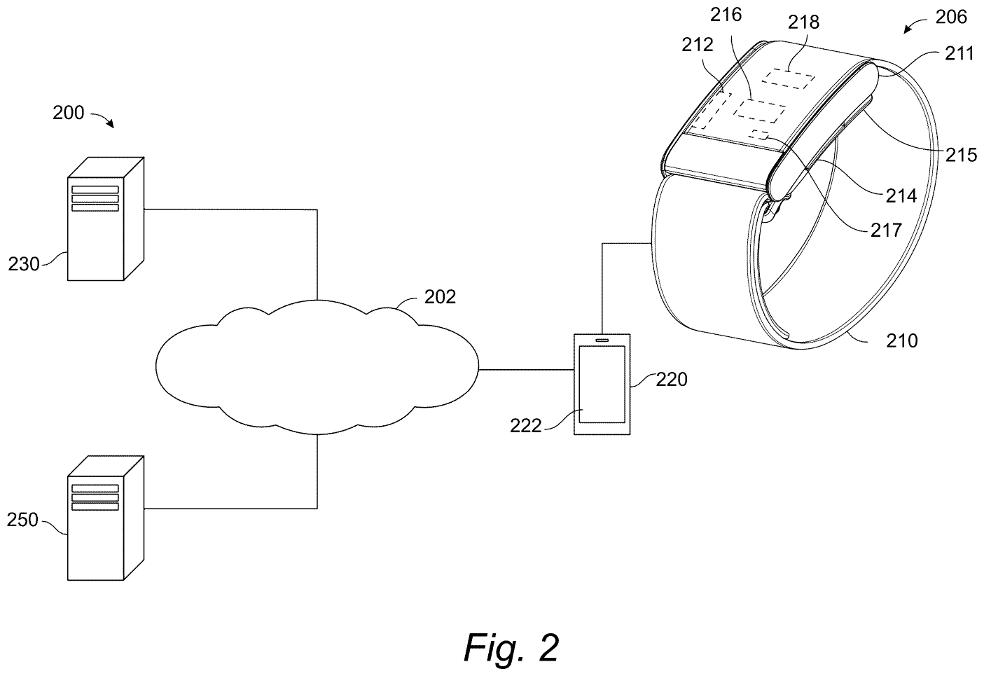

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicated detected motion, and/or beacons or the like to self-identify type, weight, and so forth, in order to facilitate automated detection and tracking of exercise activity with other connected devices.

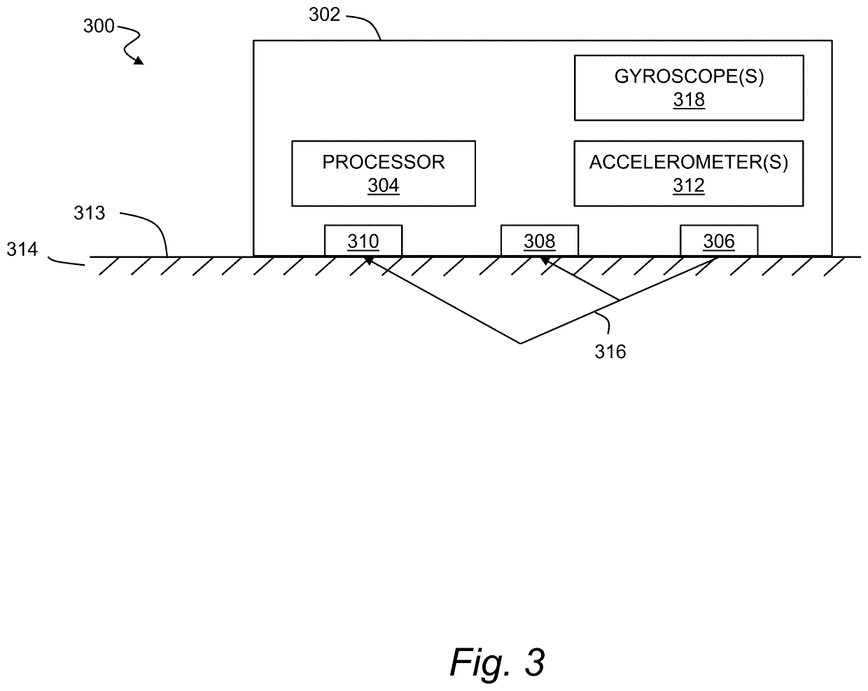

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like such as audio earbuds, earrings or similar, glasses and/or other eyewear, a ring, a headband, and so forth.

The processor 304 may be any microprocessor, microcontroller, application specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 310 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

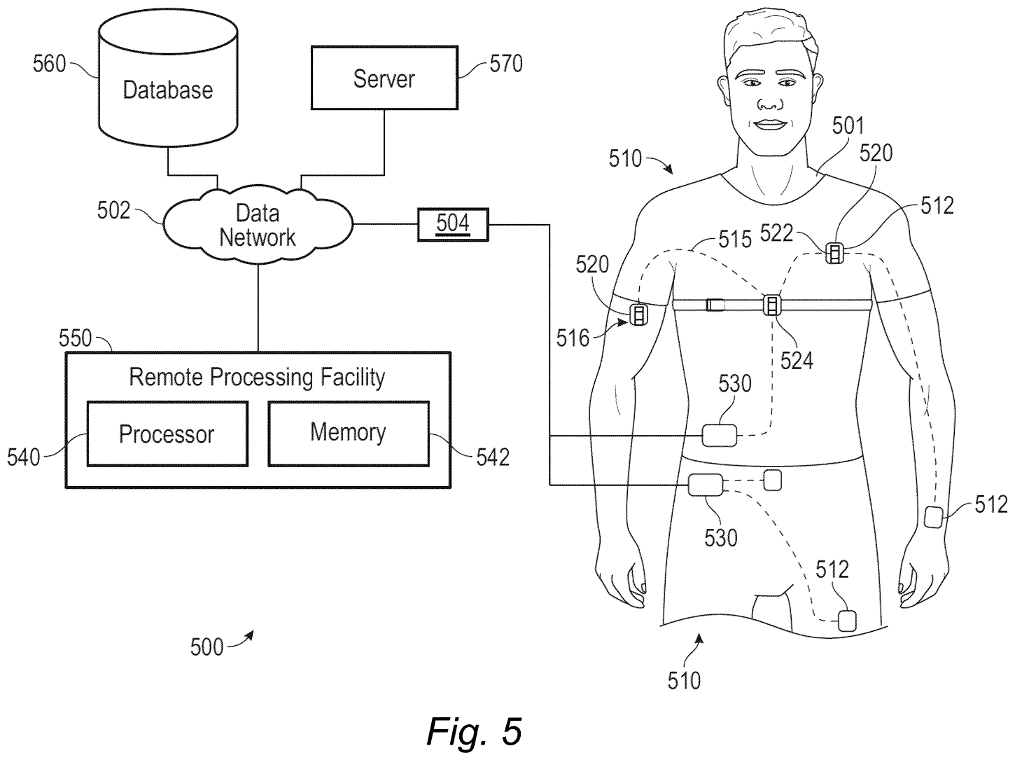

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

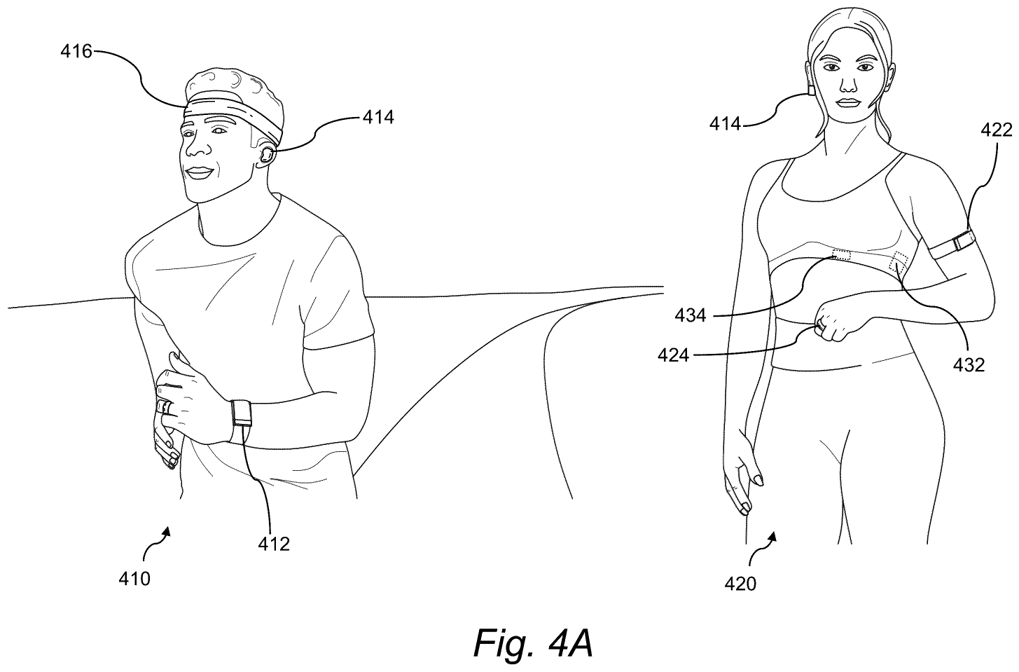

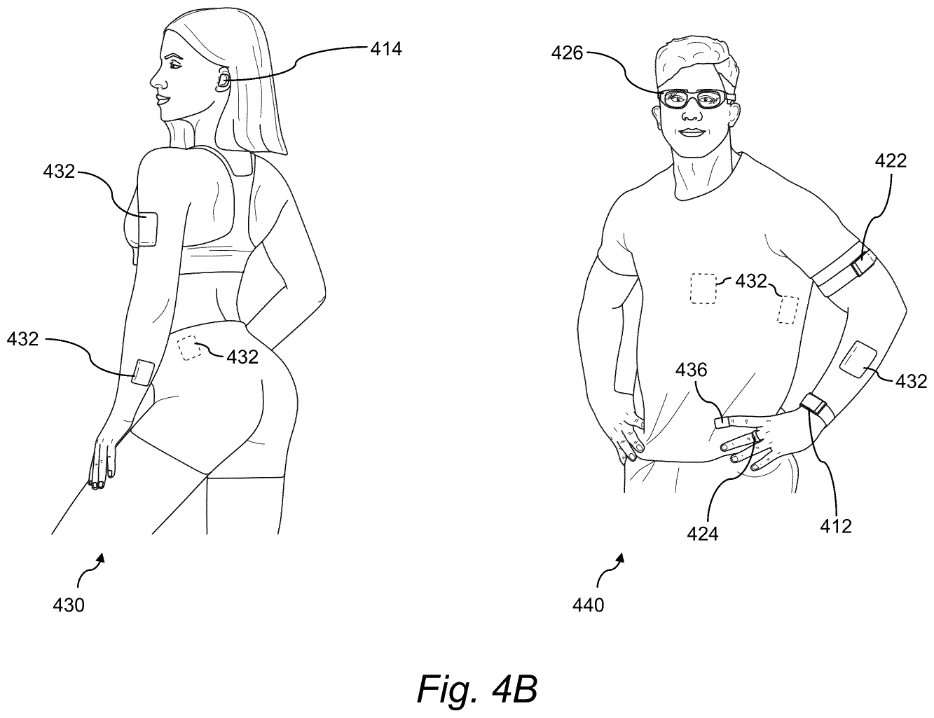

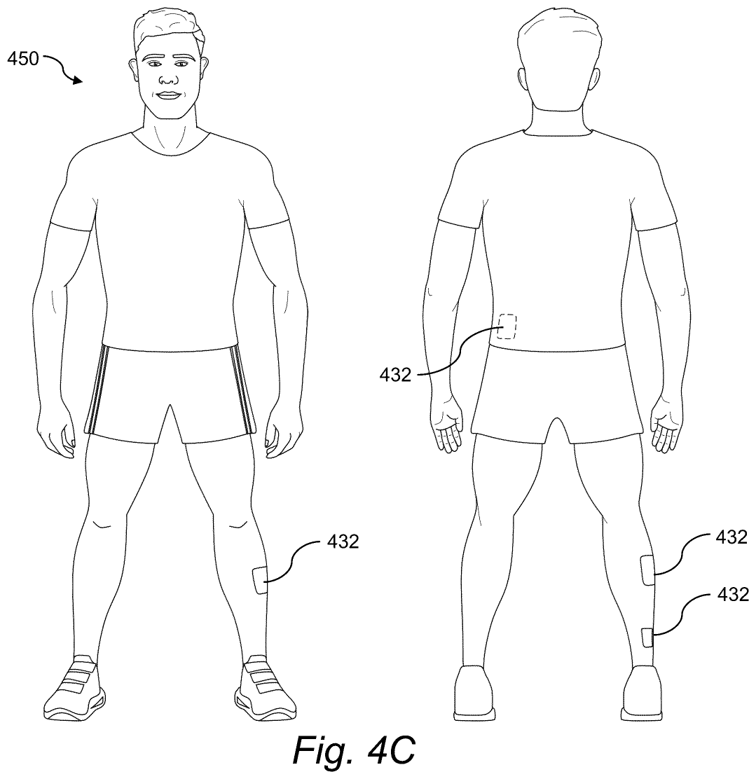

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a finger-tip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The finger-tip monitor 436 may include a clamp, clip, or the like, and may be the same or similar to any of the physiological monitors described herein. In some aspects, the finger-tip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-fi or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

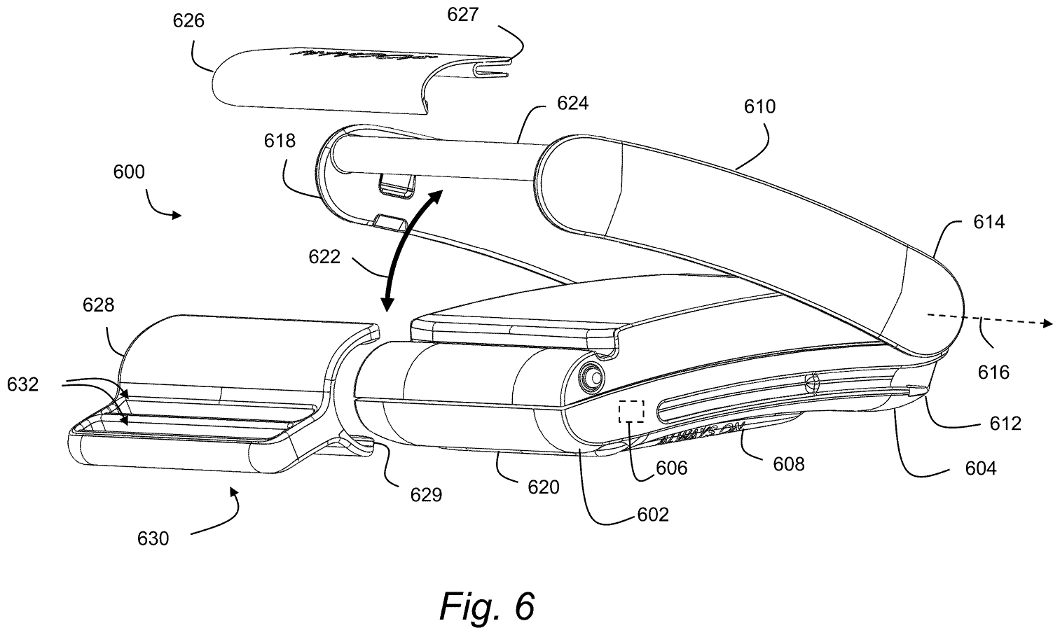

FIG. 6 shows a system 600 including a wearable monitor 602 that is structurally configured for use with an adjustable strap or band. In general, a strap, which may be any of the bands of elastic material or other straps described herein, may usefully be adjusted to a desired tension for a particular user. To this end, an adjustable strap may include a buckle on one end that removably and replaceably couples to a monitoring device while retaining a length of the strap as the buckle and strap are removed from the monitoring device and replaced. This advantageously permits a number of straps to be used interchangeably without requiring readjustment of the strap length each time a strap is changed. It will be understood that the strap may also or instead include two bands that are removably and replaceably couplable to one another, and where each band is removably and replaceably couplable to a monitoring device—e.g., a strap with bands that function similar to a watch band or the like. For example, in an aspect, such a strap includes a first band with a sliding buckle structurally configured for engaging both the band and the monitoring device, and the strap includes a second band with a clasp structurally configured for engaging both the band and the monitoring device, where the first band is engageable with the second band via a securement fixture such as a post that engages with one or more holes in one or more of the bands.

The wearable monitor 602 may be any device configured to monitor physiological data, such as a wearable physiological monitoring device that uses photoplethysmography (PPG) or the like to substantially continuously monitor heart rate variability (HRV) and the like, or any other device described herein. The wearable monitor 602 may include a housing 604 containing sensing circuitry 608 and a battery 606 within an interior space of the housing 604. The housing 604 may enclose the battery 606 and sensing circuitry 608 in a substantially waterproof enclosure, e.g., that prevents ingress of water in harmful quantities during immersion in water to at least one meter for at least thirty minutes. The term “waterproof” as used herein shall include (but is not necessarily be limited to) waterproof as specified in international standards such as the Ingress Protection (IP) rating system. For example, “waterproof” as used herein may include waterproof as specified in IP67 (i.e., dust-tight and water-resistant to a depth of 1 meter for 30 minutes) or IP68 (i.e., dust-tight and water resistant to a depth of 1.5 meters for up to 30 minutes). While these are generally accepted standards for water resistance, other standards or specifications, including more rigorous standards and specifications, may also or instead be used.

The system 600 may include a clasp 610 pivotally mounted to a first end 612 of the wearable monitor 602 on a first end 614 of the clasp 610 where there is a rotation axis 616. A second end 618 of the clasp 610 may be rotatable between a first position adjacent to a second end 620 of the wearable monitor 602 and a second position away from the second end 620 of the wearable monitor 602 as generally illustrated by an arrow 622. The clasp 610 may be rotatable around the rotation axis 616 over an angle of 180 degrees or more. The clasp 610 may include a cross member 624 on the second end 618 of the clasp 610 having an axis aligned to the rotation axis 616 for the clasp 610.

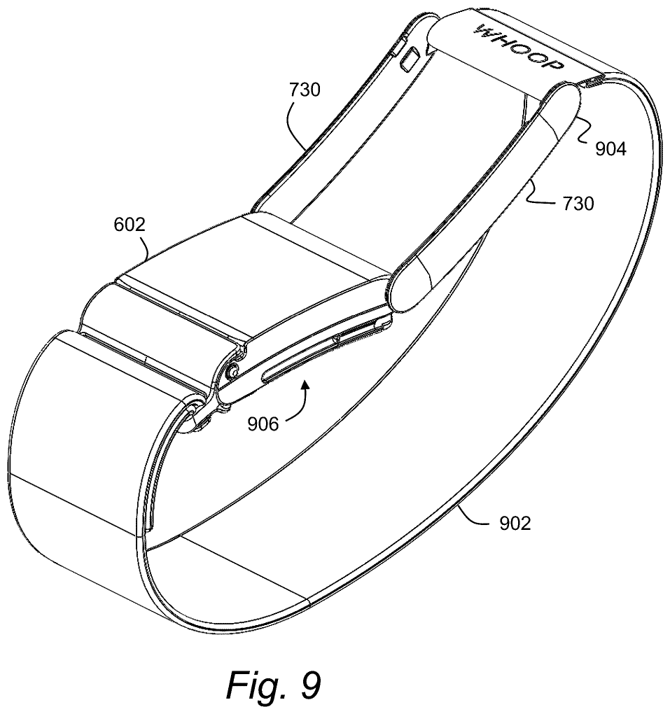

The wearable monitor 602 may be secured by a strap 902 (see, e.g., FIG. 9), which may include any of the adjustable bands or other straps described herein and may generally secure the wearable monitor 602 in a desired location on a user's body—e.g., on an appendage of a user, such as the user's wrist. The strap 902 may include a band of elastic material with a first end and a second end to provide a combination of tension to secure the wearable monitor 602 for physiological monitoring and elasticity to accommodate diameter changes resulting from user movement. The length of the band from the first end to the second end may be sufficient to wrap around a variety of body parts and accessories. In this manner, the strap 902 may accommodate a variety of wearer sizes and shapes, as well as physical movements by the wearer. The elastic material of the band may include any material designed to resist permanent deformation such as rubber, nylon, synthetic fiber, or the like. In general, the strap 902 may interconnect a hook 626 and a buckle 628, each of which may be coupled to the wearable monitor 602 and the strap 902 as described herein. In one aspect, the strap 902 may include a high friction material on a surface contacting the wearable monitor 602 when the clasp 610 is in the first position adjacent to a second end 620 of the wearable monitor 602. This can help to secure the strap 902 against the wearable monitor 602 and prevent lateral or lengthwise slippage.

The hook 626 may be crimped, adhered, or otherwise attached to the strap. In one aspect, the hook 626 may be coupled to the strap in a non-adjustable manner, e.g., crimped or otherwise affixed to a first end 614 of the strap 902. The buckle 628 may be attached to the strap 902 in a manner that permits adjustment of a position of the buckle 628 along the strap 902 in order to adjust a length of the strap 902, and a corresponding tension of the strap 902 about a wrist or other body part of a user. The buckle 628 may, for example, provide a fixture 630 defining an overlapping path for adjustably retaining a length of a band of elastic material between the buckle 628 and the hook 626. In this embodiment, the strap 902 may be woven through two adjacent slits 632 along the overlapping path through the fixture 630 to secure the buckle 628 at a desired position along the strap 902. The fixture 630 may also or instead include one or more posts or the like that are engageable (e.g., insertable) into a loop formed on the strap 902. In some embodiments, the fixture 630 may be a rigid structure extending from the buckle 628. Alternatively, the fixture 630 may be collapsed to lie unobtrusively against the buckle 628. Other adjustment techniques are known in the art, which may also or instead be used to adjustably couple the buckle 628 to the strap 902. It will be understood, however, that the hook 626 may also or instead be adjustably coupled to the strap. The crimp 627 of the hook 626 may conveniently permit the hook 626 to fold against the wearable monitor 602 with a low profile that lies flush with the clasp 610, the strap 902, and/or other hardware. In one aspect, a circumferential tension along the strap 902 may help to secure the hook 626 in a rotational orientation that prevents decoupling of the hook 626 from the rotation axis 616 of the clasp 610 when the clasp 610 is in a closed position, e.g., about a wrist of a wearer.

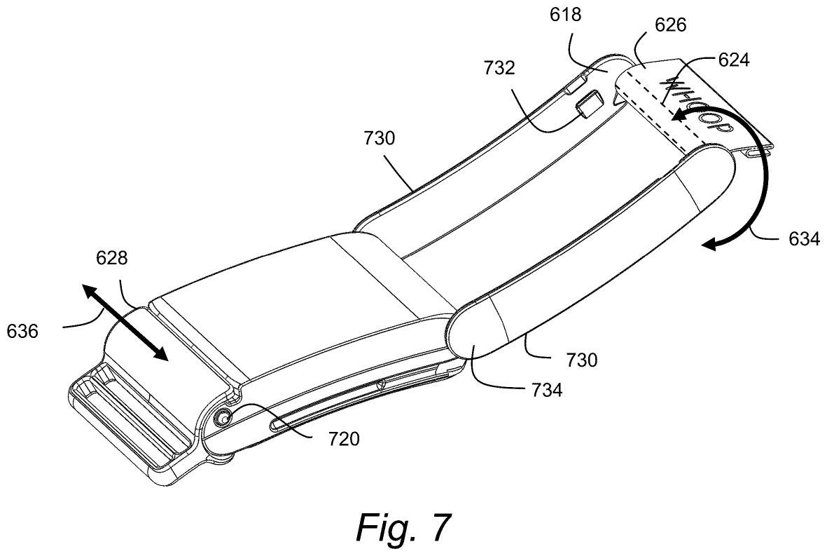

As shown in FIG. 7, the hook 626 may be rotatably coupled to the cross member 624 on the second end 618 of the clasp 610, where the hook 626 is rotatable as indicated by an arrow 634 to decouple the hook 626 from the cross member 624. Decoupling the hook 626 in this manner may prevent unwanted couplings of the hook 626 to the buckle 628 to aid in removal of the wearable monitor 602.

The buckle 628 may be linearly removable from and replaceable to the second end 620 of the wearable monitor 602 along a second axis parallel to the rotation axis 616 for the clasp 610 as indicated by an arrow 636. In this manner, the buckle 628 may be slidably engageable with the second end 620 of the wearable monitor 602. The buckle 628 may, for example, have a c-shaped cross section along the second axis shaped and sized to couple to a partially cylindrical surface on the second end 620 of the wearable monitor 602. As shown in FIG. 6, the c-shaped cross section may also include a tooth 629 or other flange or the like shaped and sized to engage an indent in the second end 620 of the wearable monitor 602 when the buckle 628 is aligned for use along the second axis. The buckle 628 may further include a fixture as described above, e.g., providing an overlapping path for adjustably retaining a length of the band of elastic material between the hook 626 and the buckle 628.

In one aspect, and as shown in FIG. 7, the wearable monitor 602 may include a spring bar 720 or similar feature with protruding surfaces that retain the clasp 610 in a closed position and prevent rotational movement of the clasp 610. The clasp 610 may include a pair of arms 730 extending from the first end 614 of the clasp 610 to the second end 618 of the clasp 610. When closed, indents 732 or the like (e.g., one or more recesses) may engage the protruding portions of the spring bar 720 to secure the clasp 610 in a closed position (or vice-versa, regarding indents/protrusions, such as where the clasp 610 includes protrusions instead of indents 732).

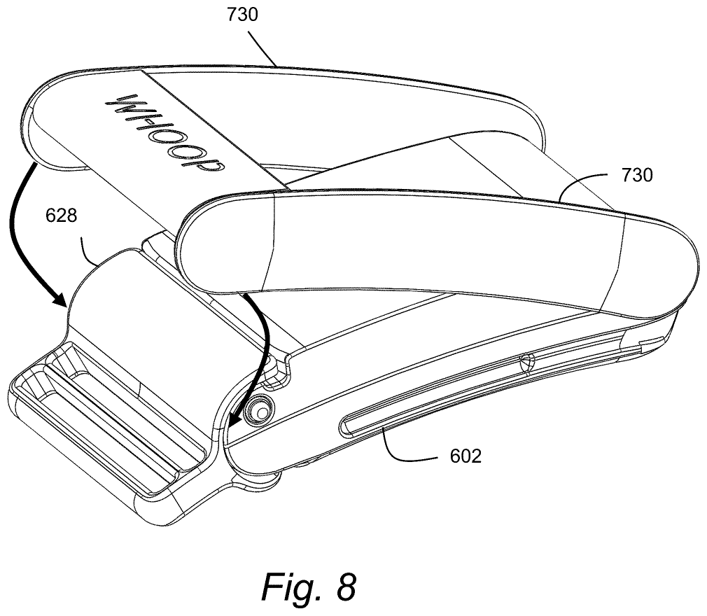

As shown in FIG. 8, when closed, the pair of arms 730 may overlap the ends of the buckle 628 in order to secure the buckle 628 against displacement along the second axis, e.g., parallel to the rotation axis 616 of the clasp 610 such as by preventing a sliding motion of the buckle 628 relative to the wearable monitor 602. The pair of arms 730 may generally rotate away from the second end 620 of the wearable monitor 602 when in a second position (or open position) to permit linear movement of the buckle 628 along the second axis to decouple the buckle 628 from the wearable monitor 602. That is, an arm 730 may be hingeably coupled to the housing 604 and rotatable around a hinge 734 of the housing 604 to releasably maintain a position of the buckle 628 and/or releasably tighten the strap in the position. And, in some aspects, the arm 730 may be removable from and replaceable to the hinge 734 of the housing 604. In certain implementations, the buckle 628 may retain a target length of the strap for a user as the buckle 628 is removed from and replaced to the wearable monitor 602, or as the user changes among different straps over time.

FIG. 9 shows a wearable monitor 602 with a strap 902. The strap 902 may be an adjustable band of elastic material configured to retain the wearable monitor 602 in a desired location, e.g., on a wrist of a user. As used herein, terms such as “strap,” “band,” “elastic band,” “adjustable band,” “adjustable strap,” and the like are used interchangeably to describe a band (or multiple bands) of elastic material or the like used to secure a physiological monitoring device to a wrist or other region of a user's body, unless a more specific meaning is otherwise provided or clear from the context. It will be further understood that, in some implementations, the band may be relatively inelastic.

In one aspect, the wearable monitor 602 may be secured to a wrist or other appendage, e.g., with the strap 902, or the like. When the wearable monitor 602 is suitably positioned, e.g., on a wrist, and suitably electromechanically structured, a user may touch a finger to an external surface of the wearable monitor 602 to form an electrical circuit through the wearable monitor 602 that facilitates the capture of an electrocardiogram (ECG) indicative of heart rate activity. In general, an ECG measures the electrical activity of the heart, which can be used to diagnose and monitor various heart conditions, such as abnormalities including atrial fibrillation, atrial flutter, ventricular tachycardia, and to suggest when a person is at risk of future heart problems. More generally, ECGs are a useful diagnostic tool in cardiology and are often used in conjunction with other tests to obtain a comprehensive understanding of a patient's heart health.

To facilitate ECG capture, the wearable monitor 602 may include a first electrode 904 (or group of one or more electrodes or similarly conductive surface(s)) on an exposed, exterior surface of the wearable monitor 602, along with a second electrode (or group of electrodes or similarly conductive surface(s)) on a bottom surface 906 thereof that is electrically isolated from the first electrode. In this configuration, a user can contact the first electrode, e.g., with a finger of an opposing hand, to complete a circuit from the first electrode to the second electrode across the chest of the user (where ECG signals originate in the heart) on one hand, and through the wearable monitor 602 on the other hand, in order to facilitate ECG monitoring with the wearable monitor 602.

The arms 730 of the wearable monitor 602 provide a convenient contact point for the first electrode 904, particularly where the arms 730 can be squeezed between the forefinger and the thumb to provide stable engagement during an ECG measurement. While the first electrode 904 is depicted as being positioned on the end of the arm 730, it will be appreciated that the entire arm 730, or any suitable portions thereof, may be conductive, and may serve as an exposed electrode 904 for the uses described herein. The arms 730 may be, by design, a removable and replaceable element of the wearable monitor 602, in order to permit replacement due to ordinary wear, and/or to facilitate replacements for aesthetic reasons, e.g., to change the color or style of the arms 730 as desired by a user. In such aspects, the wearable monitor 602 may be adapted to support a stable electrical coupling across mechanical components that are removable and replaceable, and that are also intended to move during use, e.g., by pivoting the arms 730 and any accompanying electrode contact surfaces, such as when fastening and unfastening the strap 902 of the wearable monitor 602. The design described herein may advantageously support a removable and replaceable ECG-ready contact or electrode for a wearable device integrated into a movable fastening surface. By supporting a conductive path from the removable electrode to internal, cardiography circuitry within the wearable monitor 602, the design described herein facilitates removal and replacement of system components that integrate the first electrode 904, such as a replaceable strap or band, as needed or desired by a user.

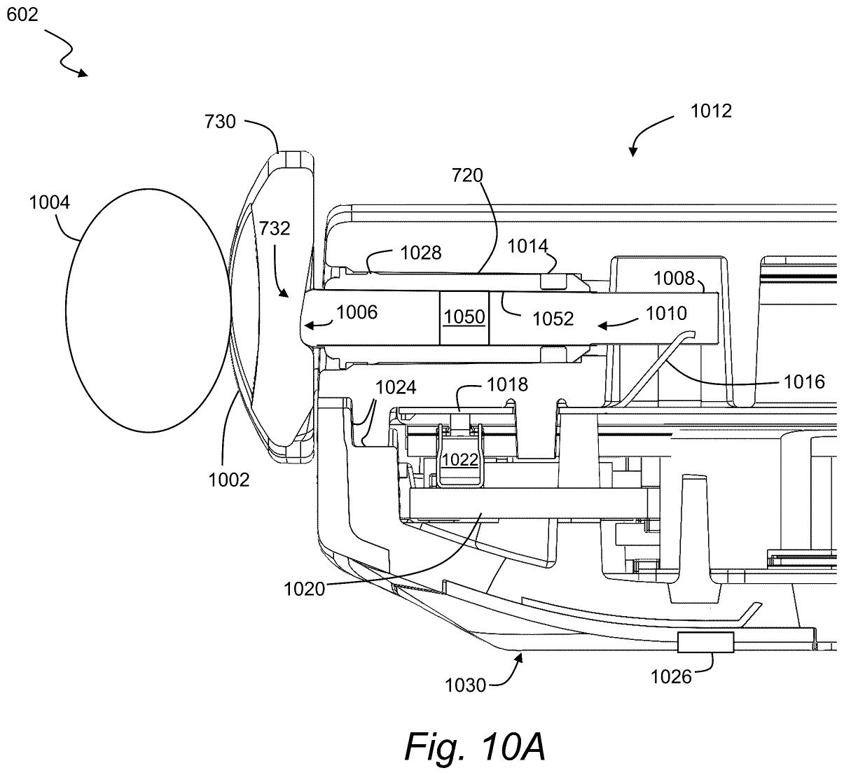

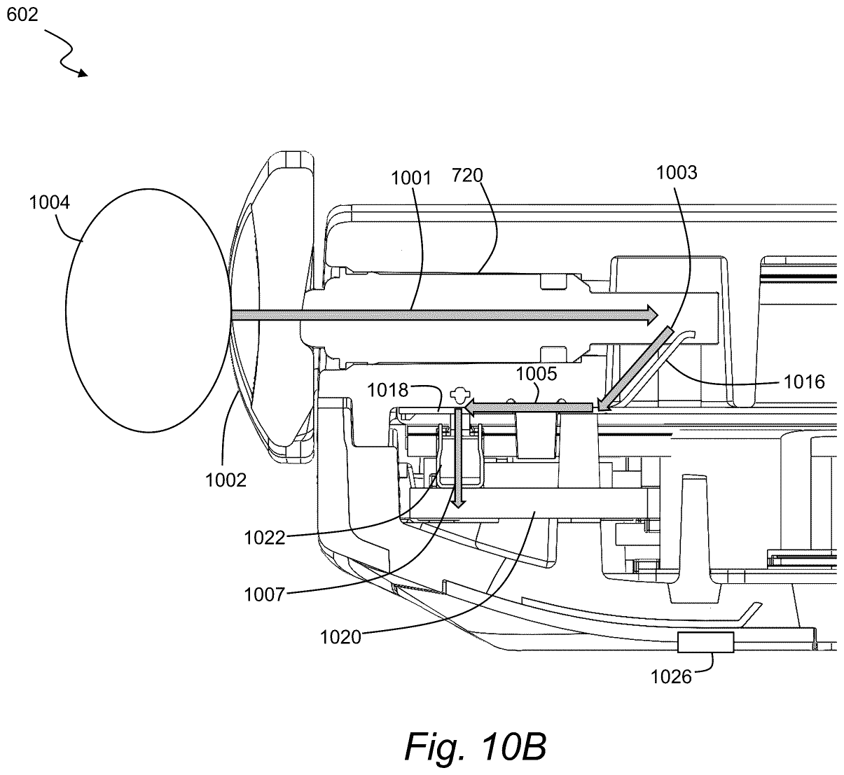

FIG. 10A shows a cross section of a wearable monitor 602, such as any of the wearable monitors described herein.

In general an arm 730 of the wearable monitor 602 may provide a first electrode 1002 formed of an electrically conductive material (and/or coating) and positioned where the first electrode 1002 can be contacted by a finger 1004 of a user to form a circuit for capturing an ECG. When the arm 730 is closed, e.g., to fasten the wearable monitor 602 about the wrist or other appendage of a user as illustrated, e.g., in FIGS. 6-9, an indent 732 on the arm 730 may contact a spring bar 720 of the wearable monitor 602. While this mechanically secures the arms 730 in a closed position to secure the strap 902 about the user, this can also concurrently form an electrical contact between an exposed surface 1006 of the spring bar 720 and the indent 732 in the arm 730. While one side of the wearable monitor 602 is depicted, it will be understood the other side (with a second spring bar) of the wearable monitor 602 may also or instead form a second electrical contact with a second arm 730, e.g., to increase conductive paths or provide a failsafe mechanism in the event that the electrical interface between the spring bar 720 and the indent 732 fails. Thus, any features shown relative to this cross-section in FIG. 10A will be understood to be potentially mirrored on an opposing side of the wearable monitor 602. Providing two opposing spring bars 720 may also or instead advantageously balance the contact force between the indents 732 and the spring bars 720 for more consistent and reliable electrical contact. One or more additional spring bars 720 with additional contacts may also be used, e.g., to increase conductivity, increase mechanical retention of the arm 730, provide additional failsafe paths, and so forth.

The conductive surface of the first electrode 1002 may be formed with a metallic material. While the arm 730 may itself be formed of a suitable metallic conductor, a variety of coatings may also or instead be used. For example, a conductive metal or oxide may be applied to a surface of the arm 730 using any suitable technique such as electroplating, electroless plating, sputtering, or the like. Physical vapor deposition advantageously permits coatings over complex surfaces in a manner that also facilitates control over texture, color, conductivity, and the like, and may usefully be employed for conductive coatings of the arm 730 of the wearable monitor 602 generally, or the first electrode 1002 specifically, for use in electrocardiography as described herein. In one aspect, the entire arm 730 may be coated with a conductive material or the like. In another aspect, specific contact regions may be selectively coated in order to control where on the external surface of the wearable monitor 602 a user makes contact to capture an ECG. The hook 626 or other components of the wearable monitor 602 may also or instead be metallized or otherwise coated to permit use in ECG capture as described herein.

The spring bar 720 may include a sleeve 1052 with a post 1008 extending from an end opposing the exposed surface 1006. In general, the post 1008 may have an electrically conductive surface, and may form a continuous electrical connection with the exposed surface 1006 to facilitate an electrical connection from the exposed surface 1006 to the post 1008 through the spring bar 720 (which may also be referred to herein as a “spring pin”). In order to seal the internal components of the wearable monitor 602 from environmental conditions, the spring bar 720 may be assembled by inserting the spring bar 720 into an opening 1010 in the housing 1012 of the wearable monitor 602, e.g., with a seal 1014 such as an O-ring or similar fixture forming a pressure seal between the spring bar 720 and the walls of the opening 1010. When the spring bar 720 is inserted into the opening 1010 during assembly of the wearable monitor 602, the post 1008 may form a sliding, electrical connection with a conductive arm 1016 or similar spring contact, moving contact, sliding contact, or the like, with the conductive arm 1016 positioned to engage the conductive surface of the post 1008 when the spring bar 720 is inserted into the opening 1010 in the housing 1012, e.g., when assembled during manufacturing.

In general, the spring bar 720 may include a number of components to facilitate the creation and maintenance of a conductive path between the first electrode 1002 on the arm 730 and the conductive arm 1016 that is sealed inside the housing 1012 after assembly, so that the first electrode 1002 can more generally form a conductive path to the printed circuit board 1020 containing electrocardiography circuitry. For example, the spring bar 720 may include one or more conductive parts forming a path between the exposed surface 1006 and the post 1008. The spring bar 720 may also include a spring 1050 urging the exposed surface 1006 toward the arm 730 (e.g., to the left in FIG. 10A) so that the spring bar 720 can mechanically secure the arm 730 in place by engaging with the indent 732 on the arm 730. It will be understood that the spring bar 720 may also incorporate a hard stop or mechanical restraint to physically limit the range of motion of the spring 1050, and to retain the spring 1050 and related components within the spring bar 720 during use. The spring 1050 may, for example, include a coil spring, leaf spring, or other spring positioned between the post 1008 and the exposed surface 1006. If an assembly forming the spring 1050 has exposed components, then the regions of exposure may preferably be positioned outside the seal 1014 (e.g., to the left of the seal 1014 in FIG. 10A) so that the post 1008 can be fully sealed within the housing 1012 by the seal 1014 to maintain a waterproof or weatherproof barrier to the inside of the housing 1012. The spring bar 720 may also or instead include a sleeve 1052, which may be any suitable material for retaining the components of the post 1008, the spring 1050, the exposed surface 1006, and so on as an integral assembly that can, in turn, be inserted into the housing and secured by the seal 1014. A flange 1028 or other retaining surfaces may be formed into an exterior surface of the sleeve 1052 in order to further mechanically retain the spring bar 720 within the housing after assembly.

The conductive arm 1016 may be coupled (mechanically and electrically) to a contact pad 1018, which forms electrical contact with a printed circuit board 1020 through a moving contact 1022 such as a leaf spring or similar contact mechanism that facilitates an electrical connection with a controlled contact force over a potentially varying linear distance. In this configuration, the housing 1012 may be manufactured as two separate halves or clam shell portions, and then welded or otherwise coupled together after assembling interior components, with the moving contact 1022 electrically bridging the connection between the two pre-assembly halves. While a variety of mechanical configurations may be used to facilitate such a two-part (or multi-part) assembly, one possible configuration is illustrated in FIG. 10A with a mechanical interface 1024 between a first half and a second half of the housing 1012. In this case, the two halves may be joined and ultrasonically welded or otherwise mechanically attached, with the moving contact 1022 accommodating variations in linear distance between the two halves (and/or components assembled to the two halves) while maintaining an electrical connection between the conductive arm 1016 and the printed circuit board 1020. It will also be appreciated that the adjoining surfaces may have a variety of contours and mating surfaces. While the specific geometry is not generally important, these contours will generally connect to form a continuous seal about the interior of the housing 1012, except, for example, where press fit pieces such as the spring bar 720, or other similar assemblies, are used to complete the environmental barrier between interior and exterior regions. Thus, for example, a two-part, waterproof clam shell assembly as contemplated herein is also facilitated by the insertable spring bar 720. For example, the two halves may be pressed together and welded under pressure. In this case, the opening 1010 for the spring bar 720 permits the escape of air, or more generally, equalization of air pressure, during the welding process. The spring bar 720 may then be inserted after assembly of the two halves, and more specifically after the components of the housing 1012 have cooled to a temperature where they are sufficiently rigid to resist deformation while the spring bar 720 is added. Additional features may support the structural integrity and environmental seal of the assembled device, such as a slight draft angle to generate an increasing friction fit as the spring bar 720 slides into the opening 1010, and/or a flange 1028 or other angled protrusion or the like to mechanically engage the spring bar 720 (or the sleeve 1052 of the spring bar 720) with the wall of the opening 1010 and resist outward movement (to the left, in FIG. 10A) after insertion.

As used herein, the term moving contact is intended to refer to any non-mechanically fixed electrical contact between two parts. For example, this may include a sliding contact through which two conductive surfaces are in sliding, electrically conductive contact with one another, such as the post 1008 and conductive arm 1016 described above. This arrangement permits an assembly of the spring bar 720 into the housing 1012 by sliding the spring bar 720 into place after the housing 1012 has been welded, epoxied, or otherwise assembled and sealed into an integral part. A moving contact may also or instead include a flexible junction such as a leaf spring or the like used as the moving contact 1022, which as described herein, permits an assembly to maintain suitable inter-component contact forces over a range of possible linear distances. In another aspect, a moving contact may include, e.g., the contact between the exposed surface 1006 of the spring bar 720 and the indent 732 of the arm 730 when the arm 730 is snapped into place over the spring bar 720, e.g., when the wearable monitor 602 is placed for use and fastened around a wrist of a user. In this case, the continuity of contact may be maintained in part by the spring force of the spring bar 720 against the indent 732 of the arm 730. More generally, any such moving, sliding, flexing, or otherwise mechanically variable contact techniques may be used to provide moving contacts as described herein, and facilitate the creation and maintenance of continuous conductive paths without fixed mechanical coupling over variable contact distances or other changing or unknown spatial relationships. Collectively, these techniques may advantageously be used to support a continuous, conductive path between a removable electrode and interior circuitry of a weatherproof housing.

In general, the printed circuit board 1020 may be coupled to a second electrode 1026 on a bottom surface 1030 of the wearable monitor 602. In this configuration, the second electrode 1026 may be in continuous contact with a user while the wearable monitor is placed for use. The printed circuit board 1020 may include ECG circuitry include electrical components and a processor (including a memory containing code) configured to acquire an electrocardiogram (ECG) based on signals measured between the first electrode 1002 and the second electrode 1026. The printed circuit board 1020 may also include code that configures the wearable monitor 602 to guide a user through a process for acquiring the ECG, or may be coupled in a communicating relationship with a user device (such as a smart phone) that guides the user through the process. In one aspect, this may include notifying a user when a circuit has been formed that is suitable for capturing an ECG signal. In another aspect, the wearable monitor 602 may be configured to opportunistically acquire an ECG when suitable conditions are satisfied (e.g., when a complete circuit through the user's opposing arm and across the chest is established). In another aspect, the wearable monitor 602 may be configured to evaluate the quality of an ECG signal that is acquired, and instruct a user to adjust conditions, e.g., by increasing or decreasing a grip pressure applied to the arms 730 with the user's fingers. In general, the techniques for acquiring, processing, and displaying an ECG signal are known in the art, and are not described here in detail.

It will be noted that, in the embodiments described above, the ECG circuit path between the electrodes is only completed when the arms 730 are closed, such that the exposed surface 1006 of the spring bar 720 is in contact with the indent 732 of the arm 730 that contains the first electrode 1002. As a significant advantage, this can help to ensure that ECG monitoring is only enabled during conditions supporting a high quality signal, e.g., when the arm 730 is closed to securely fasten the strap 902 around a wrist of a wearer.

FIG. 10B shows a cross section of a wearable monitor, such as any of the wearable monitors described herein. An electrical path for an ECG circuit is illustrated through the wearable monitor 602 with a number of arrows. The ECG circuit path may start at the body of a user, e.g., from the user's finger 1004 when engaged with the first electrode 1002 establishing a predetermined contact. From the first electrode 1002, the circuit path travels through the spring bar 720 and through a moving contact to the conductive arm 1016 as shown by the first arrow 1001. The circuit path may continue through the conductive arm 1016 to the contact pad 1018 as shown by the second arrow 1003, and through the contact pad 1018 to the moving contact 1022 as shown by a third arrow 1005. The circuit path may continue through the moving contact 1022 to the printed circuit board 1020 as shown by the fourth arrow 1007. It will be understood that the wearable monitor 602 may also include a corresponding path between the second electrode 1026 and the printed circuit board 1020, so that a second contact point on the body can be established to support ECG measurements. A variety of techniques may be used to provide a waterproof, electrical channel through the housing to the printed circuit board 1020, including the techniques described herein, as well as any other techniques suitable for a fixed, permanent electrode.

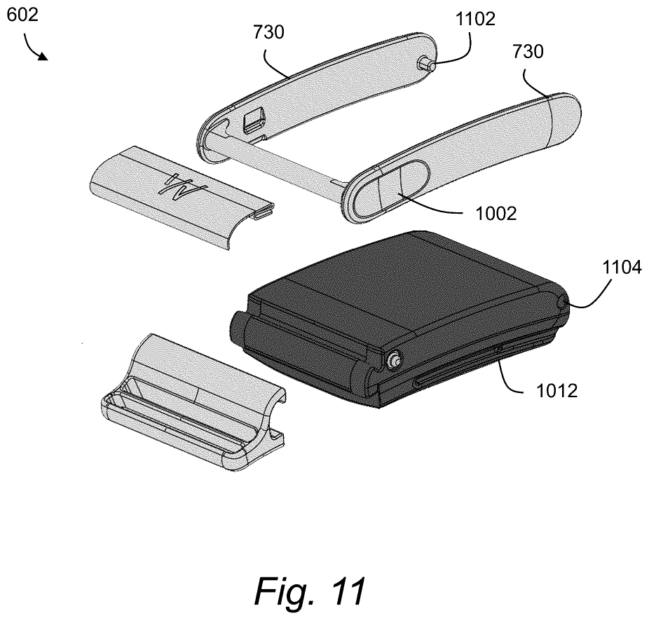

FIG. 11 shows an exploded view of a wearable monitor, such as any of the wearable monitors described herein. In general, the wearable monitor 602 may be disassembled into several components. More specifically as described herein, an arm 730 with a first electrode 1002 may be decoupled from a housing 1012 of the wearable monitor 602, e.g., by detaching the arm 730 at a hinge formed by a pair of posts 1102 in the arm 730 and corresponding holes 1104 in the housing 1012. In general, some or all of the surface of the arm 730 may be formed of a conductive material to provide the first electrode 1002. That is, the electrode 1002 may be limited to one or more specific regions of the arm(s) 730, or the entire surface of the arm(s) 730 may be conductive, and may form a part of the electrode 1002. It will also be understood that, while a hinged arm with posts is illustrated, any technique for removably and replaceably coupling the first electrode 1002 to the housing 1012 in a manner that permits an electrical circuit to be formed between the first electrode 1002 and internal electronics of the wearable monitor 602 may be used with a wearable monitor 602 as described herein.

It will be appreciated that an electrocardiogram may include one or more additional electrodes, which may reduce noise and improve signal quality and reliability.

For example, in one aspect, a three-lead ECG may include a first and second electrode as described herein, which will generally serve to detect a bioelectrical signal based on voltage differences caused by cardiac activity. In one aspect, a third electrode may provide a stable reference for the voltages acquired at the other electrodes. By actively maintaining a stable reference potential, the third electrode can reduce common-mode noise and interference, ensuring that the signals captured by the first and second electrodes are more accurate and less noisy. In one aspect, the third electrode may actively drive a common-mode voltage in order to cancel out or compensate for any common-mode interference. The third electrode may also or instead be used to mitigate interference from, e.g., skin impedance variations, motion artifacts, and the like. The improvements in signal quality from a third electrode may make electrocardiogram readings possible in high-impedance, relatively small form factor systems such as battery powered wearable devices, and can relax constraints on positioning and tightness of fit. Such a third electrode can be on the body of the housing, connected to the strap, or placed in some other location and coupled to the physiological monitoring system.

Other configurations, such as fifteen-lead, twelve-lead, six-lead are also used in a variety contexts for ECG data acquisition, and may be adapted for use in the context of, e.g., multi-sensor wearable monitoring systems such as those described herein. As a significant advantage, three-lead configurations may provide a substantially simpler electrode configuration in a more lightweight, discreet setup suitable for daily or continuous wear, while still significantly improving noise reduction due to, e.g., common mode rejection and the like.

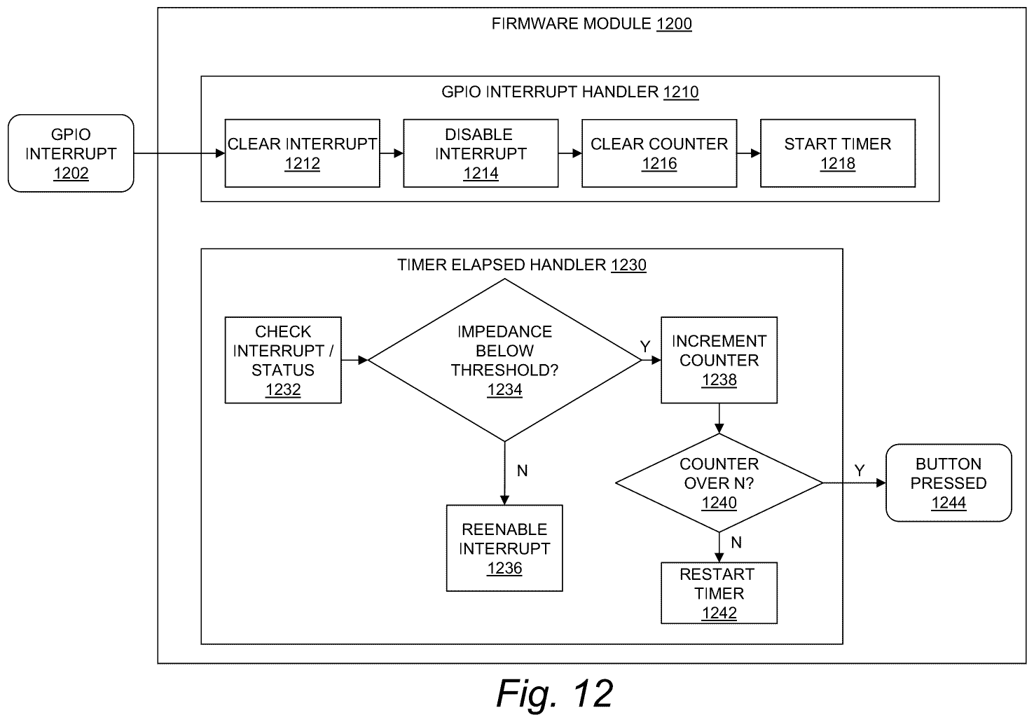

FIG. 12 shows a process for using an exposed electrode of a wearable device as a control input. In general, an exposed electrode, such as the first electrode described above for acquiring ECG data, may be used as a control input or other user interface element from a wearable monitor. This may include using a characteristic contact with the electrode as the basis for initiating an ECG scan, or this may include using some other characteristic contact as a virtual button press to initiate some other action by the wearable monitor. In general, use of the electrode as a control input requires additional processing to distinguish user intent (to use the ECG electrode as a control input) from incidental user contact or other environmental conditions that might have a similar initial electrical signature.

In general, the electrode may be any electrode on a user device, such as any of the wearable monitors described herein. The wearable monitor may include a housing with a first electrode and a second electrode, all as generally described above. The first electrode may be coupled to the second electrode through circuitry within the housing, such as electrocardiography circuitry or the like, which may sense electrical potential differences or impedance between the electrodes and process these sensed conditions accordingly. In one aspect, this may be used to acquire an electrocardiogram signal as described above. In another aspect, this may be used as a control input for operation of the device. In one aspect, the system may be configured to detect an impedance between the electrodes of less than 40 Megaohms, or some other threshold indicative of both electrodes being in contact with a user's skin. In response to this threshold, the circuitry may initiate a button detection algorithm to evaluate whether the nature and duration of the contact is indicative of a user intent to provide user input, acquire an ECG, and so forth.

As illustrated in FIG. 12, a process for using an exposed electrode as a control input may be performed by a firmware module 1200 that implements a firmware process or the like. Specifically, as shown in step 1202, a firmware process or the like may receive an interrupt, e.g., when impedance between leads is below a predetermined threshold. This interrupt may be a general-purpose I/O (“GPIO”) controller interrupt or the like, which may be configured to respond to any suitable threshold(s) for, e.g., impedance between the electrodes, with a user input detection routine. More specifically, the process may include preparing for a button detection by performing the steps shown in the GPIO interrupt handler 1210. As shown in step 1212, this may include clearing the interrupt, e.g., by acknowledging and resetting the interrupt signal so that a controller using the firmware module 1200 knows that the interrupt has been handled. As shown in step 1214, the process may include disabling the GPIO interrupt, e.g., to avoid resetting a button detection routine or executing multiple concurrent routines for button detection. As shown in step 1216, the method may include clearing a counter used to measure elapsed time. This ensures that prior counter data stored in a register or other memory location used for the counter does not interfere with a new measurement of elapsed time that is initiated by the interrupt. As shown in step 1218, once the interrupt has been handled and the counter has been reset, the process may include starting a timer.

A timer for processing electrode contact may be managed by a timer elapsed handler 1230, executing on the firmware module 1200. In general, once the interrupt has been handled by the GPIO interrupt handler 1210, the timer elapsed handler 1230 or the like may then be called, invoked, or otherwise initiated to begin measuring a duration of the low impedance state detected with the GPIO interrupt. As shown in step 1232, running the timer may include checking the interrupt (which may be the GPIO interrupt, or any other suitable interrupt for monitoring impedance between the electrodes) or a corresponding status register or the like where a current value for impedance may be stored. As shown in box 1234, running the timer may include evaluating if the impedance between the electrodes remains below the predetermined threshold (or some second threshold suitable for tracking continued contact after detecting the initial contact).

As shown in step 1236, if the impedance is no longer below the threshold, the GPIO interrupt may be re-enabled to permit a new button detection. In this case, the process may generally return to step 1202 where the process waits for another low-impedance interrupt rather than detecting a button press. As shown in step 1238, if the impedance remains below the threshold, a counter may be incremented. As shown in step 1240, the counter may then be evaluated to determine if it is over a threshold value. If the counter is over the threshold value (N), then the timer elapsed handler 1230 may report a button pressed signal, as shown in step 1244, and the device associated with the firmware module 1200 may take any suitable, responsive steps. For example, this may include capturing an ECG signal based on measurements between the electrodes, or some other user function such as changing a display of a user interface, initiating a workout, or performing some other user-initiated action. As shown in step 1242, if the counter is not over the threshold value, N, then the timer elapsed handler 1230 may be restarted by returning to step 1232 where the interrupt status is checked. In this manner, the firmware module 1200 may more generally operate in response to an initial low-impedance interrupt by either incrementing a counter while the impedance remains low until a button press is detected (step 1244), or reenabling the interrupt when the impedance rises so that a new timer can be started when appropriate. In general, the counter limit (N) and the impedance threshold may be programmatically specified, and may be tuned for a particular hardware configuration and/or user behavior to best capture user intent. That is, in some implementations, the predetermined thresholds may vary per user; in other aspects, the predetermined thresholds are fixed for all users or for some certain subset of users.

It will be understood that the GPIO interrupt, firmware module 1200, GPIO interrupt handler 1210, and timer elapsed handler 1230 may be any combination of hardware, firmware, software, and the like suitable for performing the functions described above on a device such as a wearable physiological monitor.

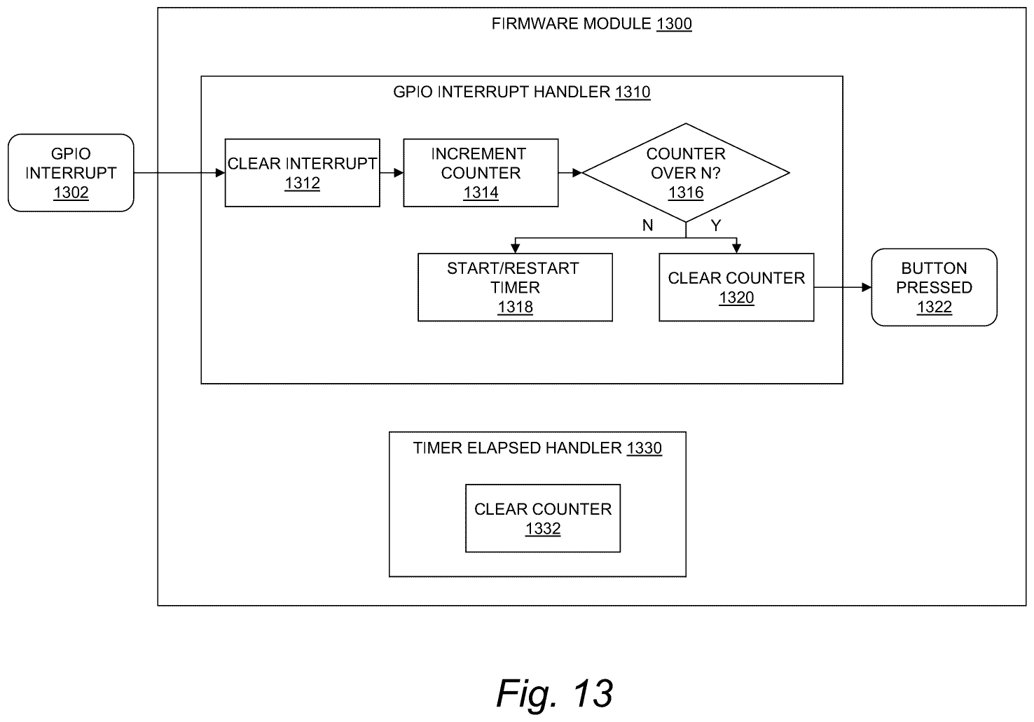

FIG. 13 shows a process for using an exposed electrode of a wearable device as a control input. In general, this may be a process similar to that described with reference to FIG. 12 above. However, in a system where the GPIO interrupt has a delay before reasserting, the hardware may not indicate that the impedance remains within the threshold the next time that the firmware checks. In order to avoid any corresponding race conditions or the like, an additional timer can be used to determine when the next interrupt should be expected for continuous contact. This approach advantageously seeks to ensure that the process will wait an appropriate amount of time for the next interrupt before determining that the button is not in use, and that there is no accidental button timeout before the next interrupt.

As shown in FIG. 13, a process for using an exposed electrode as a control input may be deployed on a firmware module 1300, and may begin as shown in step 1302, where a GPIO interrupt is received. The GPIO interrupt may be configured, for example, to respond to an impedance between a first electrode and a second electrode falling below a predetermined threshold, e.g., a threshold indicating a continuous circuit through a user between the first electrode and the second electrode. This interrupt may be configured for the system with any suitable threshold(s)), and may respond by initiating a GPIO interrupt handler 1310. As shown in step 1312, the process may include clearing the interrupt. As shown in step 1314, the counter may be incremented in response to the interrupt. As shown in step 1316, the process may include determining whether the duration is sufficient to determine that a button is being pressed for a control input, more specifically based on a threshold value, N, for the counter. As shown in step 1318, if the duration is insufficient to determine that a button is being pressed for a control input, more specifically based on a threshold value, N, for the counter, the process may including re-starting the counter.

As shown in step 1320, if the counter is above the threshold, then the counter may be cleared and, as shown in step 1322, a button pressed signal may be generated for the device. If the counter is not above the threshold, N, then a timer may be started, or if it has already started, then it may be restarted. The timer may be executed by a timer elapsed handler 1330, which may clear the counter as shown in step 1332 after a predetermined interval without a low-impedance interrupt. In this manner, the counter will only be incremented when the interrupt is received within a predetermined interval determined by the timer elapsed handler. If no interrupt is received after this amount of time, then a break in the circuit between the electrodes can be inferred, and the counter can be reset for use in a subsequent button press detection. As described above, the timer elapsed handler 1330 may be used more specifically in this example to determine how long the firmware module 1300 should wait, when trying to detect a button press, for a new interrupt after the prior interrupt has been cleared.

It will be understood that the GPIO interrupt, firmware module 1300, GPIO interrupt handler 1310, and timer elapsed handler 1330 may be any combination of hardware, firmware, software, and the like suitable for performing the functions described above on a device such as a wearable physiological monitor. It will also be understood that a variety of other techniques may be used in the context of a real time control system and/or wearable user device to monitor the contact between two sensing electrodes and provide interpretive output such as a button press signal or a request for an ECG. Any such technique may be used with the wearable devices described herein.

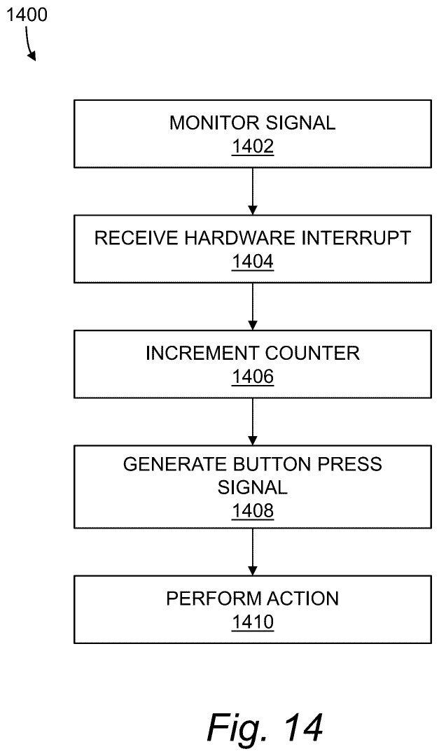

FIG. 14 is a flow chart of a method for using a physiological monitoring electrode as a user input. The method 1400 may be performed using any of the systems, devices, and techniques described herein, e.g., any of the wearable physiological monitors and/or monitoring systems described herein.

As shown in step 1402, the method 1400 may include monitoring two or more electrodes of a wearable monitor, such as a wrist-worn heart rate monitor or any of the other wearable monitors described herein. The two or more electrodes may include electrodes configured to provide a physiological signal of a user such as an electrocardiogram. As described herein, this may include a first electrode on an exposed surface of a wearable device positioned where a user can selectively contact the first electrode, e.g., by placing a finger (of a limb opposing the limb where the wearable device is secured) on the first electrode to initiate an ECG or provide a user input signal for one or more other actions by the wearable device. The two or more electrodes may also include a second electrode positioned on a surface of the wearable device that is in contact with the user's skin when the wearable device is placed for use, e.g., on a bottom surface of the wearable device that is continuously in contact with the wearer's skin.

As shown in step 1404, the method 1400 may include receiving a hardware interrupt responsive to a predetermined impedance between the first electrode and the second electrode, such as an impedance meeting a predetermined threshold indicative of user contact with the first electrode, or more generally, an electrical circuit through the user between the first electrode and the second electrode.

As shown in step 1406, the method 1400 may include, while the hardware interrupt indicates that the impedance meets the predetermined threshold, incrementing a counter. In general, the counter may be used to measure the amount of time that the impedance meets the threshold, e.g., to ensure a prolonged, continuous contact of the first electrode by the user indicating an intention to acquire ECG data.

As shown in step 1408, the method 1400 may include, in response to the counter meeting a second predetermined threshold, generating a button press signal to a process executing on the wearable monitor. The second predetermined threshold may, for example, be selected to distinguish incidental contact with the first electrode from intentional contact with the first electrode, and/or to programmatically debounce this virtual button to prevent sporadic, repetitive triggering of downstream device functions.

As shown in step 1410, the method 1400 may include receiving the button press signal with code executing on the wearable monitor, and, in response to the button press signal, performing an action responsive to a corresponding user input. This may, for example, include initiating an ECG based on a measured signal between the first electrode and the second electrode. This may also or instead include initiating some other action by the wearable device that is suitable responsive to an intentional button press by the user. This may, for example, include starting a timer, updating or changing a display on the wearable device, uploading data from the device to a remote processing resource, indicating the beginning or end of a workout, requesting a physiological measurement such as a blood pressure measurement or an oxygen saturation measurement, and so forth.

It will be understood that, while a pair of electrodes is described, the foregoing technique may also or instead be used with three or more electrodes where a threshold impedance can usefully be measured between any two or more of the electrodes in order to infer a user intention. It will also be understood that, while the counter may be updated using any of the techniques described, for example, in FIG. 12 or 13, other hardware, firmware, and/or software techniques may also or instead be used to monitor impedance between electrodes of a wearable physiological monitor and convert these into a user input to initiate an ECG, or otherwise control operation of the device. All such variations that would be understood by one of ordinary skill in the art are intended to fall within the scope of this disclosure.

In an aspect, the method 1400, and/or other processes and techniques described herein, may be implemented using a computer program product. That is, in an aspect, a computer program product may include computer executable code embodied in a non-transitory computer readable medium that, when executing on one or more processors of a wearable monitor, causes the wearable monitor to perform the steps of: monitoring a physiological signal of a user with at least two electrodes on the wearable monitor; receiving a hardware interrupt indicative of an impedance between the at least two electrodes meeting a predetermined threshold; while the hardware interrupt indicates that the impedance meets the predetermined threshold, incrementing a counter; and, in response to the counter meeting a second predetermined threshold, generating a button press signal to a process executing on the wearable monitor.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A wearable monitor comprising:
a housing containing circuitry and sensors for physiological monitoring;
a strap configured to secure the housing in a position about a wrist of a user for use in physiological monitoring;
an arm hingeably coupled to the housing and rotatable around a hinge of the housing to tighten the strap in the position about the wrist of the user, the arm including a recess;
a first electrode on an exterior surface of the arm;
a spring pin positioned to removably and replaceably secure the arm with the hinge in a closed position such that the strap is tightened in the position for use, the spring pin including an external contact surface for electromechanically coupling to the recess in the arm, and an electrical contact extending into an interior of the housing, the electrical contact of the spring pin conductively coupled to the external contact surface of the spring pin;
a printed circuit board within the housing, the printed circuit board electrically coupled to the electrical contact of the spring pin through one or more moving contacts;
a second electrode on the housing, the second electrode coupled to the printed circuit board, and the second electrode positioned to contact a skin of the user when the housing is in the position for use by the user during physiological monitoring; and
electrocardiogram circuitry on the printed circuit board, the electrocardiogram circuitry coupled between the first electrode and the second electrode, and the electrocardiogram circuitry configured to capture an electrocardiogram of the user when a user contacts the first electrode with a digit on a second arm opposing a first arm where the housing is secured to the wrist by the strap.

2. The wearable monitor of claim 1, wherein the arm is removable from and replaceable to the hinge of the housing.

3. The wearable monitor of claim 1, wherein the housing is waterproof.

4. The wearable monitor of claim 1, wherein the printed circuit board is electrically coupled to the electrical contact of the spring pin through at least one sliding contact.

5. The wearable monitor of claim 1, wherein the printed circuit board is electrically coupled to the electrical contact of the spring pin through at least one leaf spring.

6. The wearable monitor of claim 1, wherein the printed circuit board is electrically coupled to the electrical contact of the spring pin through at least one moving contact.

7. The wearable monitor of claim 1, further comprising photoplethysmography circuitry configured to acquire heart rate data from the user through photoplethysmography when the wearable monitor is placed for use on the user.

8. A wearable monitor comprising:
a housing with an exterior surface and an interior space;
electrocardiogram circuitry in the interior space of the housing;
a first electrode, wherein:
the first electrode is positioned for access by a user when the wearable monitor is placed for use on the user, and
the first electrode is removably and replaceably coupled to the housing; and
a second electrode, wherein:
the second electrode is positioned on the exterior surface of the housing,
the second electrode is positioned to contact a skin of the user when the wearable monitor is placed for use on the user, and
the second electrode is coupled to the first electrode through the electrocardiogram circuitry in the housing.

9. The wearable monitor of claim 8 wherein the wearable monitor includes a photoplethysmography monitor.

10. The wearable monitor of claim 8, further comprising a strap for securing the housing to the user.

11. The wearable monitor of claim 10, further comprising a fastener configured to releasably tighten the strap to retain the wearable monitor for use on the user.

12. The wearable monitor of claim 11, wherein the first electrode is positioned on a surface of the fastener.

13. The wearable monitor of claim 11, wherein the fastener and the strap are removable from and replaceable to the housing.

14. The wearable monitor of claim 11, wherein the fastener has an open position and a closed position, and further wherein:
in the open position of the fastener, the wearable monitor is removable from the user and the first electrode is not electrically coupled to the electrocardiogram circuitry, and
in the closed position, the strap retains the housing of the wearable monitor against the skin of the user for acquisition of a physiological signal and the first electrode is electrically coupled to the electrocardiogram circuitry.

15. The wearable monitor of claim 10, wherein the strap includes two bands that are removably and replaceably couplable to one another.

16. A computer program product comprising computer executable code embodied in a non-transitory computer readable medium that, when executing on one or more processors of a wearable monitor, causes the wearable monitor to perform the steps of:
monitoring two or more electrodes of a wearable monitor, wherein:
the two or more electrodes including a first electrode and a second electrode,
the second electrode is in continuous contact with a skin of a user of the wearable monitor when the wearable monitor is placed for use on the user, and
the wearable monitor is configured to acquire electrocardiographic data with the two or more electrodes;
receiving a hardware interrupt with the wearable monitor, the hardware interrupt responsive to a predetermined impedance between the first electrode and the second electrode, and the predetermined impedance indicative of user contact with the first electrode;
while the hardware interrupt indicates the predetermined impedance between the first electrode and the second electrode, periodically incrementing a counter; and
in response to the counter meeting a second predetermined threshold, generating a button press signal to a process executing on the wearable monitor.

17. The computer program product of claim 16, wherein the wearable monitor includes a wrist-worn heart rate monitor.

18. The computer program product of claim 16, further comprising code that, in response to the button press signal, causes the wearable monitor to perform a responsive action.

19. The computer program product of claim 16, further comprising code that, in response to the button press signal, causes the wearable monitor to initiate an electrocardiograph acquisition.

20. The computer program product of claim 16, wherein the second predetermined threshold for the counter is selected to distinguish intentional contact with the first electrode by the user from unintentional contact with the first electrode.
